(19) United States
(12) Patent Application Publication
(10) Pub. No.: US 2021/0046201 A1
(43) Pub. Date: Feb. 18, 2021

(54) UV LINE BEAM STERILIZER

(71) Applicant: KOREA ELECTRO-OPTICS CO., LTD., Incheon (KR)

(72) Inventors: Dong Jin HAN, Incheon (KR); Myung Seop KIM, Seoul (KR); Se Jin BAEK, Bucheon-si, Gyeonggi-do (KR); Sang Su HA, Hongseong-gun, Chungcheongnam-do (KR)

(73) Assignee: KOREA ELECTRO-OPTICS CO., LTD., Incheon (KR)

(21) Appl. No.: 16/991,778

(22) Filed: Aug. 12, 2020

(30) Foreign Application Priority Data
Aug. 13, 2019 (KR) 10-2019-0098652

(51) Int. Cl. A61L 2/10; A61L 2/24

(52) U.S. Cl. CPC A61L 2/10; A61L 2/24; A61L 2202/20; A61L 2202/14; A61L 2202/11

(57) ABSTRACT

An ultraviolet (UV) line beam sterilizer may include a UV light source unit including upper and lower light source modules and radiating UV line beams to top and bottom surfaces of personal effects being moved through a conveyor, a reflection plate unit including left and right reflection plates radiating UV line beams to left and right surfaces of the personal effects by reflecting the UV line beams from the upper and lower light source modules, and a wedge-shaped reflection plate radiating UV line beams to the front and back of the personal effects by reflecting the UV line beams, a sensor unit including sensors installed at intervals in a row on the conveyor and outputting sensing signals for moved locations of the personal effects, and a controller outputting a pulse width modulation signal and a switching control signal based on the sensing signals and user setting information.

### BACKGROUND

### 1. Technical Field

The present disclosure relates to a technology for sterilizing an object being moved through a conveying unit using ultraviolet (UV) rays, and more particularly, to a UV line beam sterilizer capable of sterilizing colon bacilli, Vibrio ordalii or viruses rapidly and in large quantities by radiating UV line beams to surfaces of personal effects while moving the personal effects through conveying means, such as a conveyor, in a school, a facility or public institution in which people having weak immune systems are accommodated, such as a sanatorium, or a communal living facility, such as a military camp.

### 2. Related Art

Recently, as an interest in personal hygiene is gradually growing, a UV sterilizer is actively researched and developed. Personal effects related to personal hygiene management include a mobile phone, toys, a portable medical device, etc. Among them, particularly, the mobile phone is most touched by people throughout the day, and requires sterilization treatment due to a severe level of contamination. Moreover, sterilization treatment needs to be periodically performed on the mobile phone by taking bacterial growth and infections into consideration.

A storage type UV sterilizer for radiating UV rays using a mercury lamp is widely used as a sterilizer for personal effects according to a conventional technology. The conventional UV sterilizer sterilizes articles using UV rays radiated from the mercury lamp, that is, a light source, within a box type storage box.

Furthermore, another conventional UV sterilizer has disadvantages in that an article being moved through a conveyor, that is, a sterilization target, is not uniformly sterilized because uniform UV rays are not radiated to the articles and a conveyor belt perforated in a wire mesh form must be used to sterilize the bottom of the moved article. Furthermore, the conventional UV sterilizer has an inefficient problem in that sterilization must be performed for a long time, for example, several tens of minutes or several hours because a sterilizing power is weak.

An example of a UV sterilizer according to a conventional technology includes Korean Patent No. 1000438570000. The conventional UV sterilizer has a difficulty in immediately sterilizing articles that are loaded onto a conveyor and moved because it requires a warm-up time of about 10 minutes to 1 hour in order to obtain light energy of a given value or more.

Recently, a light-emitting diode (LED), a laser diode (LD), etc. have been developed and in the spotlight as alternative light sources of the mercury lamp. Particularly, the LED light source has advantages in that the LED light source has a small size, can be used without a warm-up time by selecting only a desired wavelength band, and has tens of thousands of hours in lifespan and is eco-friendly.

However, the LED light source has a disadvantage in that the price of an LED that emits light of a UV band (e.g., 250 to 280 nm) having an excellent sterilizing power is high. For example, an LED that emits UV rays of a 405 nm band has a very lower price than a light source that emits UV rays of a 250 to 280 nm (UV-C) band, but has a sterilizing power about 1,000 times weaker than the light source. Accordingly, the LED is chiefly used as the light source of a sterilizer for sterilizing articles which can be sterilized for a long time.

As described above, the UV sterilizer using the LED light source according to the conventional technology is applied to the storage type sterilizer because UV rays must be radiated for several minutes to several hours due to the insufficient amount of light and uniform sterilization is difficult. A conventional UV sterilizer that uses the LED light source and is applied to a conveyor has problems in that sterilization efficiency thereof is low and UV rays harmful to the human body leak to the outside because the conventional UV sterilizer is implemented to directly radiate UV rays to articles to be sterilized without passing through an optical system.

### SUMMARY

Various embodiments are directed to uniformly sterilizing personal effects within a short time using a relatively small number of UV light sources and reflection plates, when sterilizing and treating all of the surfaces of the personal effects using a UV line beam while conveying the personal effects through a conveyor in a public place where the prevention of epidemics is necessary.

Also, various embodiments are directed to blocking UV rays from leaking to the outside when sterilizing personal effects using a UV light source and a reflection plate.

In an embodiment, a ultraviolet (UV) line beam sterilizer may include a UV light source unit including an upper light source module and a lower light source module and configured to radiate UV line beams to top and bottom surfaces of personal effects being moved through a conveyor, a reflection plate unit including left and right reflection plates configured to radiate UV line beams to left and right surfaces of the personal effects by reflecting the UV line beams radiated from the upper light source module and the lower light source module, respectively, and a wedge-shaped reflection plate configured to radiate UV line beams to front and back surfaces of the personal effects by reflecting the UV line beams, a sensor unit including a plurality of sensors installed at intervals in a row on a carry-in part conveyor and carry-out part conveyor of the conveyor and configured to output corresponding sensing signals by detecting the locations of the moved personal effects, a controller configured to output a pulse width modulation signal and a switching control signal based on the sensing signals and setting information of a user, and a motor driving unit configured to drive a motor for the conveyor and rotate the wedge-shaped reflection plate to a reflection location or a non-reflection location by driving a motor for the reflection plate in response to the switching control signal.

### BRIEF DESCRIPTION OF THE DRAWINGS

FIG. 1 is a block diagram of a UV line beam sterilizer according to an embodiment of the present disclosure.

FIG. 2 is an external perspective view of a conveyor to which the UV line beam sterilizer according to an embodiment of the present disclosure is applied.

FIG. 3 is a perspective view of a conveyor to which the UV line beam sterilizer according to an embodiment of the present disclosure is applied.

FIGS. 4A and 4B are detailed diagrams of upper and lower light source modules according to an embodiment of the present disclosure.

FIG. 5 is an explanatory diagram for describing the output and reflection of UV line beams in the upper and lower light source modules according to an embodiment of the present disclosure.

FIGS. 6A to 6C are explanatory diagrams for describing the reflection principle of a wedge-shaped reflection plate according to an embodiment of the present disclosure.

FIGS. 7A and 7B are explanatory diagrams for describing an operation of a shutter for UV blocking according to an embodiment of the present disclosure.

FIGS. 8A and 8B are explanatory diagrams for describing an operation of the wedge-shaped reflection plate according to an embodiment of the present disclosure.

FIG. 9 is a flowchart illustrating a UV sterilization treatment operation of the UV line beam sterilizer according to an embodiment of the present disclosure.

### DETAILED DESCRIPTION

Exemplary embodiments will be described below in detail with reference to the accompanying drawings.

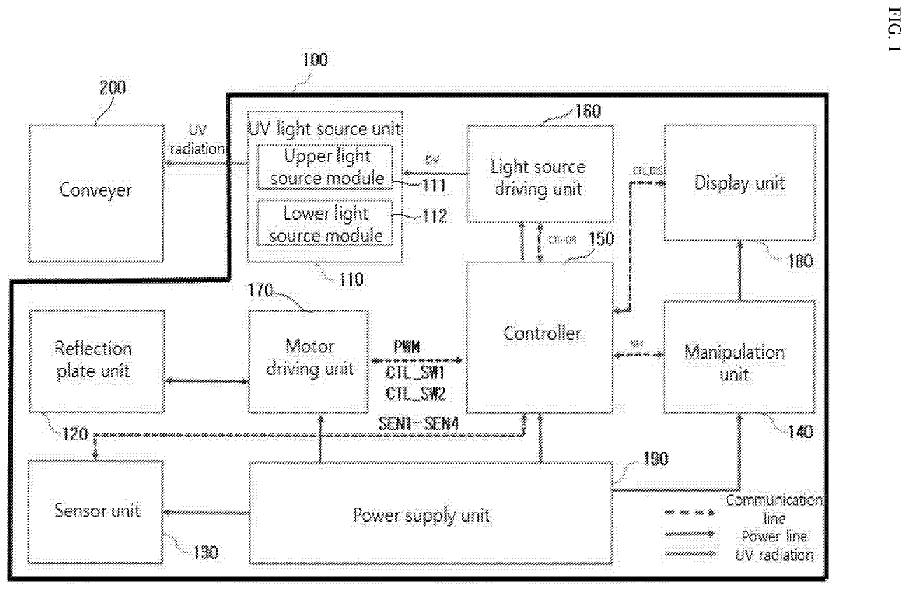

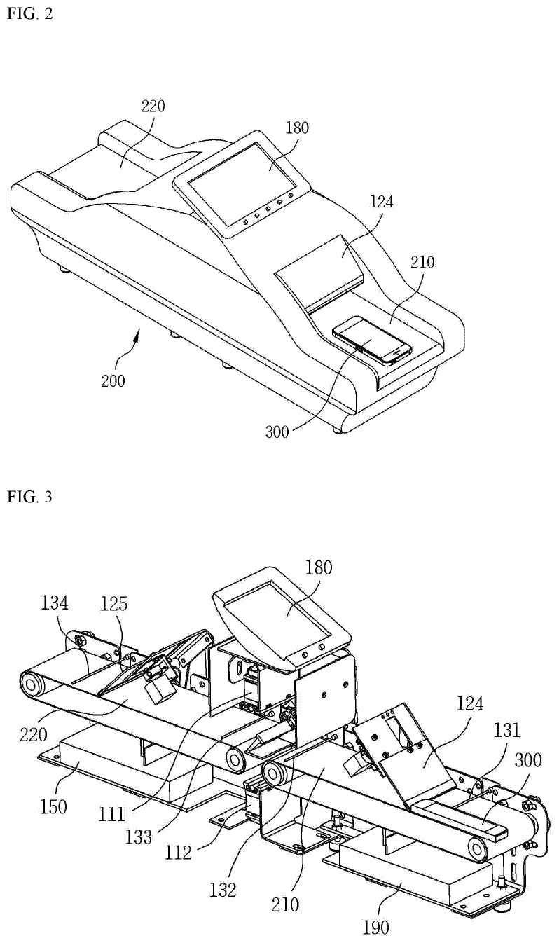

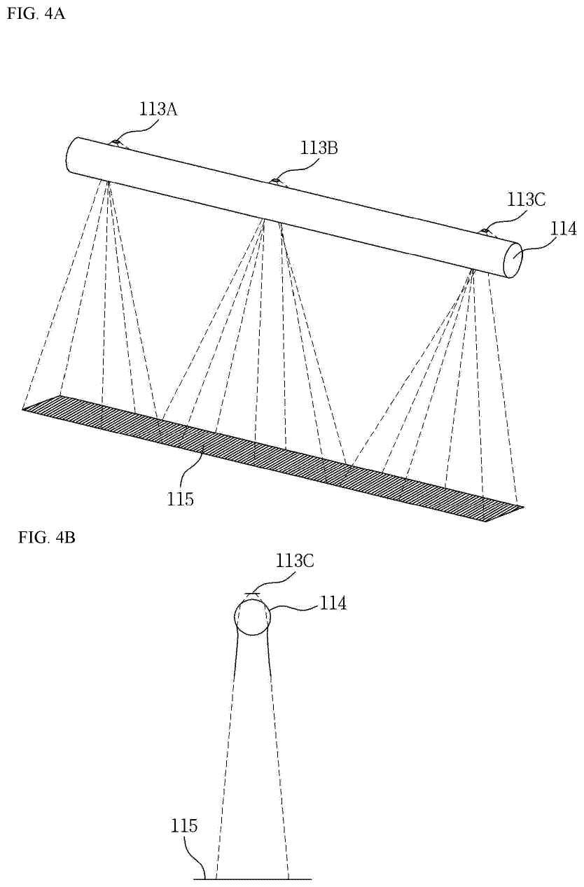

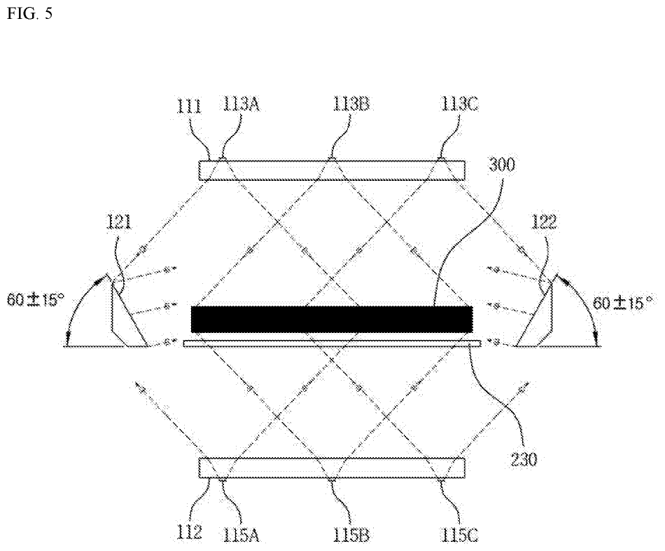

FIG. 1 is a block diagram of a UV line beam sterilizer 100 according to an embodiment of the present disclosure. FIG. 2 is an external perspective view of a conveyor to which the UV line beam sterilizer 100 according to an embodiment of the present disclosure is applied. FIG. 3 is a perspective view of a conveyor to which the UV line beam sterilizer 100 according to an embodiment of the present disclosure is applied. FIGS. 4 to 8 are partial detailed diagrams of the UV line beam sterilizer 100 according to an embodiment of the present disclosure.

Referring to FIG. 1, the UV line beam sterilizer 100 according to an embodiment of the present disclosure includes a UV light source unit 110, a reflection plate unit 120, a sensor unit 130, a manipulation unit 140, a controller 150, a light source driving unit 160, a motor driving unit 170, a display unit 180 and a power supply unit 190.

The UV line beam sterilizer 100 directly radiates UV line beams or radiates reflected UV line beams to all the surfaces of personal effects 300 that are being moved through a conveyor 200 in order to sterilize and treat the personal effects 300. The personal effects 300 may include a mobile phone, toys, a portable medical device, etc. In the present embodiment, a mobile phone is described as an example of the personal effects 300.

The conveyor 200 includes a carry-in part conveyor 210 and a carry-out part conveyor 220 installed at a given interval. A material for the conveying surfaces of the carry-in part conveyor 210 and the carry-out part conveyor 220 is not specially limited, but may include a soft material, such as urethane, rubber or plastic. A transmissive window 230 is positioned between the carry-in part conveyor 210 and the carry-out part conveyor 220. The transmissive window 230 functions to prevent the personal effects 300 from downward dropping when the personal effects 300 pass between the carry-in part conveyor 210 and the carry-out part conveyor 220 and to transmit a UV line beam radiated from the lower light source module 112 so that the UV line beam is radiated to the bottom surface of the personal effects 300.

The UV light source unit 110 radiates UV line beams, having a radiated light form, to the top and bottom surfaces of the personal effects 300 that is being moved through the conveyor 200. To this end, the UV light source unit 110 includes an upper light source module 111 positioned over the transmissive window 230 and a lower light source module 112 positioned under the transmissive window 230. The upper light source module 111 and the lower light source module 112 have the same structure and function. The type of light source used in the upper light source module 111 and the lower light source module 112 is not specially limited. In the present embodiment, an LED light source for radiating a UV line beam in a surface form is taken as an example.

Referring to FIG. 4, the upper light source module 111 includes one or more LEDs 113A to 113C and a round rod lens 114. The round rod lens 114 functions to convert light, radiated from the LEDs 113A to 113C, into a line beam in a surface form and to output the converted light.

That is, the round rod lens 114 functions to uniformly spread a Gaussian beam that is surface-emitted and incident from the LEDs 113A to 113C and to elongate the beam in one direction. The round rod lens 114 transmits an incident beam by horizontally extending the beam and vertically reducing the width of the beam in a parallel light form. If more LEDs are arranged in the length direction of the round rod lens 114 in parallel, a beam can become more uniform in a longitudinal direction. In this case, in general, curvature of the round rod lens 114 may be a spherical surface or a paraboloid or a non-spherical surface.

The distance between the LEDs 113A to 113C and the round rod lens 114 is set as a given value (e.g., 2 mm) or less in order to minimize a loss of energy spreading to the outside of the round rod lens 114. Furthermore, the distance between the round rod lens 114 and a beam radiation surface 115 may maintain a given value (e.g., 40 mm) so that the surfaces of the personal effects 300 can be relatively uniformly sterilized and treated by reducing beam spreading.

A material for the round rod lens 114 is not specially limited. However, common glass having a very low UV transmission factor is not used as the material. Sapphire, Pyrex, fused silica, quartz (SiO₂), or chloride series, that is, fluoride series (CaF₂, MgF₂) capable of transmitting UV rays, may be used as the material for the round rod lens 114. The material for the round rod lens 114 has a low refractive index of 1.4 to 1.5 and a maximum transmission factor of about 90%. Silica or quartz may transmit even vacuum UV (VUV) although it is different depending on quality. UV-fused silica has advantages in that it has a high transmission factor from UV rays to near-infrared rays, has excellent processing precision, and is the most frequently used as a material for UV rays due to an excellent environment effect according to a temperature change. The UV grade-fused silica material is used in all lenses for the UV light source unit 110 by taking the advantages into consideration. The lenses have a refractive index of 1.46 and Abbe's number of 67.8 with respect to UV rays having a 265 nm wavelength. Furthermore, the lenses are non-reflection coated, and thus have reflectance of about 1.0% or less for a UV wavelength in a UV-C wavelength band. Furthermore, the lenses may be non-reflection coated with a high refraction material (e.g., ZrO₂) and a low refraction material (e.g., SiO₂) having a good UV transmission factor. In such a case, the transmission factor is improved.

Energy density of a UV-C wavelength radiated from the UV light source unit 110 is not specially limited and is 40 mW/cm²/sec in the present embodiment. According to experiment results, it was found that most of colon bacilli and Vibrio ordalii were sterilized if energy output of a UV line beam is 10 mW/cm²/sec and most of viruses were sterilized if energy output of a UV line beam is 100 mW/cm²/sec.

Furthermore, a UV line beam radiated from the UV light source unit 110 has a size of about 90 mm in a length direction and a width of 10 mm. In such a case, experiment results showed that colon bacilli and Vibrio ordalii in an area of 50 mm×90 mm per second could be sterilized. This corresponds to an output capable of sterilizing a mobile phone within about 3 seconds.

The light sources for the upper light source module 111 and the lower light source module 112 are not specially limited. For example, the light source may have a wavelength of 278 nm, output of 0.1 W, a size of 1×1 mm, and a beam divergence angle of several tens of degrees or more.

The size, location and direction of a UV line beam radiated from each of the upper light source module 111 and the lower light source module 112 may be adjusted by adjusting the installation location of each of the upper light source module 111 and the lower light source module 112. Each of the upper light source module 111 and the lower light source module 112 may be additionally arranged in series in accordance with the size of the personal effects 300.

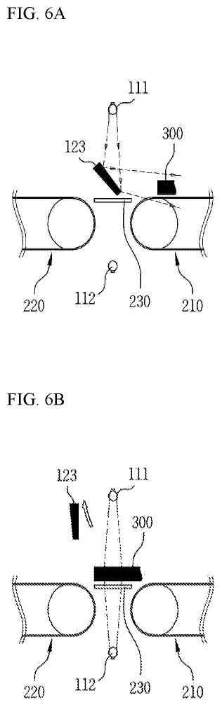

Accordingly, as in FIG. 5, a UV line beam radiated from the upper light source module 111 is directly radiated to and sterilizes the top surface of the personal effects 300. A UV line beam radiated from the lower light source module 112 is radiated to and sterilizes the bottom surface of the personal effects 300 through the transmissive window 230.

The reflection plate unit 120 includes a left reflection plate 121, a right reflection plate 122 and a wedge-shaped reflection plate 123. As in FIG. 5, the left reflection plate 121 reflects a left UV line beam, radiated from the upper light source module 111, at a given angle (e.g., 60±15°) so that the left surface of the personal effects 300 is radiated and sterilized by the left UV line beam. The right reflection plate 122 reflects a right UV line beam, radiated from the upper light source module 111, at the given angle so that the right surface of the personal effects 300 is radiated and sterilized by the right UV line beam. The wedge-shaped reflection plate 123 functions to radiate a UV line beam, radiated from the upper light source module 111, to the front part of the personal effects 300 by reflecting the UV line beam, and functions to radiate a UV line beam, radiated from the lower light source module 112, to the rear part of the personal effects 300 by reflecting the UV line beam.

Glass or metal may be used as a material for the left and right reflection plates 121 and 122 and wedge-shaped reflection plate 123 of the reflection plate unit 120. Each of the left and right reflection plates 121 and 122 and wedge-shaped reflection plate 123 may have a flat panel shape or a right-angled prism shape. In such a case, if the intensity of a light source incident on the lenses is great, the intensity of the light source needs to be taken into consideration because the metallic material may be deformed by heat unlike the glass material. The reflection plate may be coated with aluminum in order to minimize a loss of a UV beam. If the reflection plate is coated with a dielectric multi-layer thin film, reflectance of UV rays can be further increased.

The sensor unit 130 includes first to fourth photo sensors 131 to 134. The first photo sensor 131 generates a corresponding first sensing signal SEN1 by detecting that the personal effects 300 reach a carry-in location on the carry-in part conveyor 210. The second photo sensor 132 generates a corresponding second sensing signal SEN2 by detecting that the personal effects 300 deviate from the carry-in location. The third photo sensor 133 generates a corresponding third sensing signal SEN3 by detecting that the personal effects 300 is carried in the carry-out part conveyor 220. The fourth photo sensor 134 generates a corresponding fourth sensing signal SEN4 by detecting that the personal effects 300 are carried in the end part of the carry-out part conveyor 220.

The manipulation unit 140 has a user interface function for transmitting setting information SET so that a user may set a sterilization time, sterilization energy, the start and the end when the surfaces of the personal effects 300 are sterilized and treated using the UV line beam sterilizer 100 by radiating a UV line beam to the surface. The manipulation unit 140 may be implemented as a physical button or may be implemented in the display unit 180 in a touch pad form.

The controller 150 outputs a driving control signal CTL_DR, a pulse width modulation signal PWM, and first and second switching control signals CTL_SW1 and CTL_SW2 based on the first to fourth sensing signals SEN1 to SEN4 generated by the sensor unit 130 and the setting information SET output by the manipulation unit 140, and outputs a display control signal CTL_DIS for displaying the number of personal effects 300, a UV use time, and an energy intensity state based on the output and state information OUT/STATE provided from the UV light source unit 110. The controller 140 may use serial communication, such as RS-232 and RS-485, or other communication methods in order to output the signals. For example, the controller 140 is supplied with the first to fourth sensing signals SEN1 to SEN4 in real time through serial peripheral interface (SPI) communication with the sensor unit 130. The controller 140 outputs the driving control signal CTL_DR and the pulse width modulation signal PWM based on the first to fourth sensing signals SEN1 to SEN4 generated by the sensor unit 130 and the transport time of the personal effects 300 on the conveyor 200.

The light source driving unit 160 outputs driving power DV to the UV light source unit 110 in response to the driving control signal CTL_DR generated by the controller 140 so that a UV line beam having corresponding output is output.

The motor driving unit 170 drives a motor for the conveyor in response to the pulse width modulation signal PWM generated by the controller 150 so that the carry-in part conveyor 210 and carry-out part conveyor 220 of the conveyor 200 travel at a corresponding speed. Furthermore, the motor driving unit 170 controls the driving of a motor 171 for a pair of shutters in response to the first switching control signal CTL_SW1 so that shutters 124 and 125 for blocking UV rays are open or closed. Furthermore, the motor driving unit 170 drives a motor for the reflection plate in response to the second switching control signal CTL_SW2 so that the wedge-shaped reflection plate 123 switches to a reflection location or a non-reflection location.

The display unit 180 displays the number of personal effects 300 that have been sterilized and treated, a UV light source use time, and energy intensity and state in response to the display control signal CTL_DIS generated by the controller 140.

The power supply unit 190 supplies power to the elements of the UV line beam sterilizer 100, that is, the UV light source unit 110, the reflection plate unit 120, the sensor unit 130, the manipulation unit 140, the controller 150, the light source driving unit 160, the motor driving unit 170, and the display unit 180.

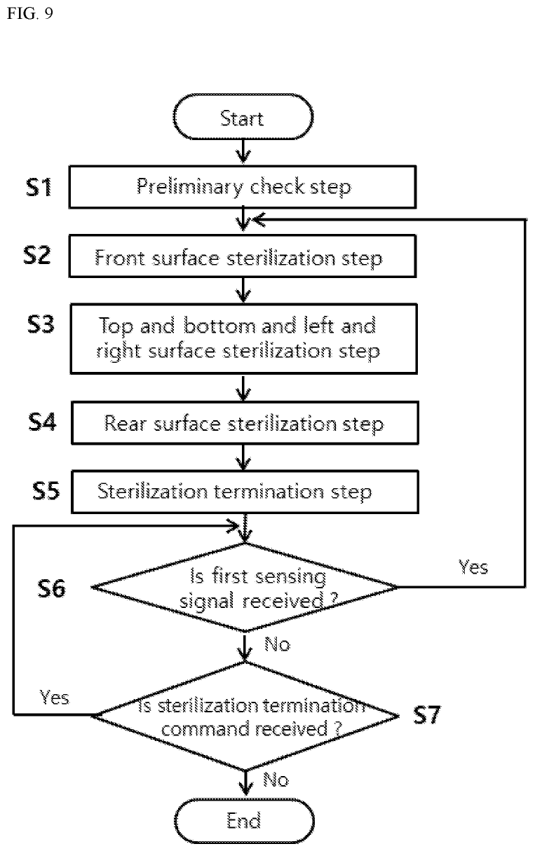

FIG. 9 is a flowchart illustrating a UV sterilization treatment operation of the UV line beam sterilizer 100 according to an embodiment of the present disclosure. A sterilization and treatment process of the UV line beam sterilizer 100 is described below with reference to FIG. 9.

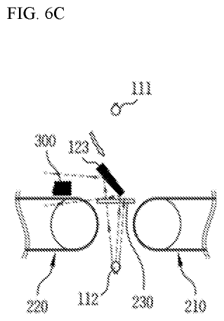

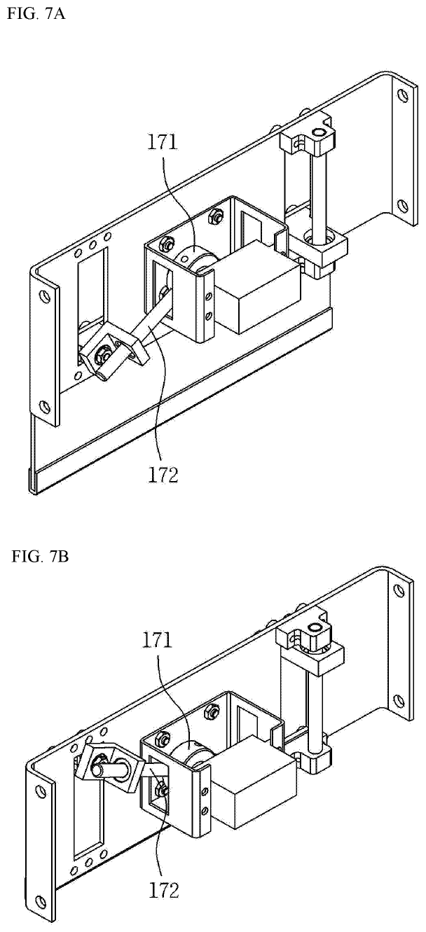

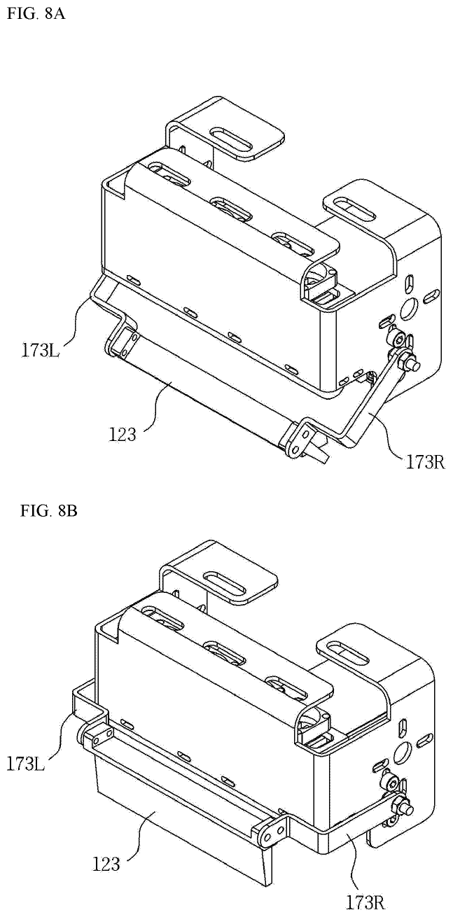

First, when a user turns on the power switch of the power supply unit 190, the controller 150 performs a preliminary check step S1. The preliminary check step may include an operation of checking, by the controller 150, whether a system operates normally, for example, the step of checking whether the conveyor 200 operates normally, by driving the motor for the conveyor using the motor driving unit 170 and checking whether the UV light source unit 110 operates normally, by driving the UV light source unit 110 using the light source driving unit 160. Furthermore, the preliminary check step may further include the step of checking, by the controller 150, that a user sets a low-speed sterilization mode, a high-speed sterilization mode, an expert mode and a sterilization condition setting mode through the manipulation unit 140. After checking that the first sensing signal SEN1 is received from the sensor unit 130, the controller 150 performs a front surface sterilization step S2 on the personal effects 300. At this time, the controller 150 outputs the pulse width modulation signal PWM to the motor driving unit 170 to drive the motor so that the carry-in part conveyor 210 and the carry-out part conveyor 220 are driven. Furthermore, the controller 150 outputs the first switching control signal CTL_SW1 of “High” to the motor driving unit 170 so that the motor 171 for the shutters is forward driven. Accordingly, as in FIG. 7A, arms 172 for the shutters are downward rotated to close the shutters 124 and 125 for blocking UV rays. Furthermore, the controller 150 outputs the second switching control signal CTL_SW2 of “High” to the motor driving unit 170 so that the motor for the reflection plate is forward driven. Accordingly, as in FIGS. 6A and 8A, a pair of arms 173L and 173R for the reflection plate coupled to the driving shaft of the motor for the reflection plate is downward rotated, so that the wedge-shaped reflection plate 123 is moved to the reflection location. Furthermore, the controller 150 outputs the driving control signal CTL_DR to the light source driving unit 160, so that a UV line beam is radiated from the upper light source module 111. Accordingly, a UV line beam radiated from the UV light source unit 110 is not radiated to the outside because it is blocked by the shutters 124 and 125 for blocking UV rays and covers near the shutters. Furthermore, as in FIG. 6A, a UV line beam radiated from the upper light source module 111 is reflected at a given angle (e.g., 45°) by the wedge-shaped reflection plate 123, so that the UV line beam is radiated to the front part of the personal effects 300 being carried through the carry-in part conveyor 210.

After checking that the second sensing signal SEN2 is received from the sensor unit 130, the controller 150 performs a top and bottom and left and right surface sterilization step S3 on the personal effects 300. At this time, the controller 150 outputs the second switching control signal CTL_SW2, corresponding to a “low” position, to the motor driving unit 170, so that the wedge-shaped reflection plate 123 is moved to the non-reflection location as in FIGS. 6B and 8B. Accordingly, when the personal effects 300 are placed on the transmissive window 230 as in FIG. 6B, the top surface of the personal effects 300 is sterilized by a UV line beam radiated from the upper light source module 111, and the bottom surface of the personal effects 300 is sterilized by a UV line beam radiated from the lower light source module 112. At this time, the left and right surfaces of the personal effects 300 are simultaneously sterilized by the left reflection plate 121 and the right reflection plate 122, respectively.

After checking that the third sensing signal SEN3 is received from the sensor unit 130, the controller 150 performs a rear surface sterilization step S4 on the personal effects 300. At this time, the controller 150 outputs the second switching control signal CTL_SW2, corresponding to a “high” position, to the motor driving unit 170, so that the wedge-shaped reflection plate 123 is moved to the reflection location as in FIGS. 6C and 8A. Accordingly, as in FIG. 6C, a UV line beam radiated from the lower light source module 112 is reflected by the wedge-shaped reflection plate 123 and radiated to the rear part of the personal effects 300 being carried out through the carry-out part conveyor 220.

After checking that the fourth sensing signal SEN4 is received from the sensor unit 130, the controller 150 performs a sterilization termination step S5 on the personal effects 300. At this time, the controller 150 stops the driving of the lower light source module 112 by blocking the driving control signal CTL_DR being supplied to the light source driving unit 160. Furthermore, the controller 150 backward drives the motor 171 for the shutter by outputting the first switching control signal CTL_SW1 of “Low” to the motor driving unit 170. Accordingly, as in FIG. 7B, the arms 172 for the shutters are upward rotated to open the shutters 124 and 125 for blocking UV rays, respectively. Furthermore, the controller 150 stops the driving of the carry-in part conveyor 210 and the carry-out part conveyor 220 by blocking the pulse width modulation signal PWM.

In such a state, when determining that the first sensing signal SEN1 is received again, by checking whether the first sensing signal SEN1 is supplied again (S6), the controller 150 repeatedly performs the second step S2 to the fifth step S5 as described above in order to sterilize other personal effects 300 using UV rays. Furthermore, the controller 150 increases an operation number count value by 1 whenever the second step S2 to the fifth step S5 are performed, and stores the increased count value.

If, as a result of the check at step S6, the first sensing signal SEN1 is not received and a sterilization termination command is received from a user, however, the controller 150 terminates the series of sterilization treatment processes (S7).

The UV line beam sterilizer according to an embodiment of the present disclosure has an effect in that it can sterilize and treat personal effects with uniformly high output within a short time using a smaller number of light sources, without being specially influenced by the sizes or shapes of the personal effects, by directly sterilizing and treating the top and bottom surfaces of the personal effects using UV line beams and sterilizing and treating the remaining surfaces of the personal effects using the reflection plate, when sterilizing and treating all of the surfaces of the personal effects using UV line beams while moving the personal effects on a conveyor in a public place where the prevention of epidemics is necessary.

While various embodiments have been described above, it will be understood to those skilled in the art that the embodiments described are by way of example only. Accordingly, the disclosure described herein should not be limited based on the described embodiments.

What is claimed is:

1. An ultraviolet (UV) line beam sterilizer comprising:
a UV light source unit comprising an upper light source module and a lower light source module and configured to radiate UV line beams to top and bottom surfaces of personal effects being moved through a conveyor;
a reflection plate unit comprising left and right reflection plates configured to radiate UV line beams to left and right surfaces of the personal effects by reflecting the UV line beams radiated from the upper light source module and the lower light source module, respectively, and a wedge-shaped reflection plate configured to radiate UV line beams to front and back of the personal effects by reflecting the UV line beams;
a sensor unit comprising a plurality of sensors installed at intervals in a row on a carry-in part conveyor and carry-out part conveyor of the conveyor and configured to output corresponding sensing signals by detecting moved locations of the personal effects;
a controller configured to output a pulse width modulation signal and a switching control signal based on the sensing signals and setting information of a user; and
a motor driving unit configured to drive a motor for the conveyor and rotate the wedge-shaped reflection plate to a reflection location or a non-reflection location by driving a motor for the reflection plate in response to the switching control signal.

2. The UV line beam sterilizer of claim 1, wherein light sources for the upper light source module and the lower light source module are light-emitting diodes (LED).

3. The UV line beam sterilizer of claim 1, wherein each of the upper light source module and the lower light source module comprises:
one or more LEDs configured to output UV line beams; and
a round rod lens configured to uniformly spread a Gaussian beam surface-emitted and incident from the LEDs and elongate the beam in one direction.

4. The UV line beam sterilizer of claim 3, wherein:
the one round rod lens is installed or a plurality of the round rod lenses is installed, and
the plurality of round rod lenses is arranged in parallel.

5. The UV line beam sterilizer of claim 1, further comprising a transmissive window positioned between the carry-in part conveyor and carry-out part conveyor of the conveyor.

6. The UV line beam sterilizer of claim 5, wherein:
the wedge-shaped reflection plate is positioned over the transmissive window,
when the personal effects are moved through the carry-in part conveyor, the wedge-shaped reflection plate radiates, to the front of the personal effects, a UV line beam radiated from the upper light source module by reflecting the UV line beam, and
when the personal effects are moved through the carry-out part conveyor, the wedge-shaped reflection plate radiates, to the back of the personal effects, a UV line beam radiated from the lower light source module, by reflecting the UV line beam.

7. The UV line beam sterilizer of claim 1, wherein:
the wedge-shaped reflection plate is rotated by the motor for the reflection plate driven under control of the controller, and
the wedge-shaped reflection plate is rotated to the reflection location of UV line beams, radiated from light sources for the upper light source module and the lower light source module, or rotated to deviate from the reflection location, by a pair of arms for the reflection plate coupled to a driving shaft of the motor for the reflection plate.

8. The UV line beam sterilizer of claim 1, further comprising a shutter for blocking UV rays, which is installed in each of an entrance and exit of the conveyor and operates in a shutter manner in order to prevent UV line beams, radiated from the upper light source module and the lower light source module, from leaking to the outside.

9. The UV line beam sterilizer of claim 1, wherein the sensor unit comprises:
a first photo sensor configured to output a corresponding first sensing signal by detecting that the personal effects reach a carry-in location on the carry-in part conveyor;
a second photo sensor configured to output a corresponding second sensing signal by detecting that the personal effects deviate from the carry-in location;
a third photo sensor configured to output a corresponding third sensing signal by detecting that the personal effects are carried in the carry-out part conveyor; and
a fourth photo sensor configured to output a corresponding fourth sensing signal by detecting that the personal effects are carried in an end part of the carry-out part conveyor.

10. The UV line beam sterilizer of claim 1, wherein the personal effects comprise any one of a mobile phone, toys, and a portable medical device.
